(19) United States
(12) Patent Application Publication
(10) Pub. No.: US 2024/0416046 A1
(43) Pub. Date: Dec. 19, 2024

(54) SUPPLEMENTARY DEVICE WITH INTEGRATED LOCKING MECHANISM

(71) Applicant: SANOFI, Paris (FR)

(72) Inventors: Stefan Alt, Frankfurt am Main (DE); Tim Glasser, Rüsselsheim (DE); Michael Helmer, Frankfurt am Main (DE); Stephan Mücke, Rüsselsheim (DE); Peter Nober, Rüsselsheim (DE); Michael Schabbach, Frankfurt am Main (DE); Martin Vitt, Frankfurt am Main (DE)

(21) Appl. No.: 18/706,044

(22) PCT Filed: Nov. 1, 2022
(86) PCT No.: PCT/EP2022/080417

§ 371 Date: Apr. 30, 2024

(30) Foreign Application Priority Data
Nov. 3, 2021 (EP) 21315221.8

(51) Int. Cl. A61M 5/315; A61M 5/172

(52) U.S. Cl. CPC A61M 5/31546; A61M 5/172

(57) ABSTRACT

The present disclosure relates to a supplementary device for attaching to a drug delivery device, wherein the drug delivery device is operable to set and to dispense a dose of a medicament, the supplementary device comprising a fastener configured to fasten to a first component of the drug delivery device, a control element configured to engage with a second component of the drug delivery device, the control element being movable relative to the fastener for inducing a movement of the second component relative to the first component when the fastener is attached to the first component, an electronic unit 70 comprising a processor, a locking mechanism comprising an electromechanical actuator controllable by the processor and operable to block or to impede a movement of the control element relative to the fastener.

### CROSS-REFERENCE TO RELATED APPLICATIONS

The present application is the national stage entry of International Patent Application No. PCT/EP2022/080417, filed on Nov. 1, 2022, and claims priority to Application No. EP 21315221.8, filed on Nov. 3, 2021, the disclosures of which are incorporated herein by reference.

### TECHNICAL FIELD

The present disclosure relates to a supplementary device for use with a drug delivery device.

The disclosure further relates to a drug delivery system.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, e.g. injection devices and needle based injection system (NIS) devices, such as pen-type injectors, have to meet a number of user-specific conditions. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

For example, a patient suffering from a particular disease may require a certain amount of a medicament to either be injected via a pen-type injection syringe or infused via a pump.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, e.g., the medicament, has been used up.

With reusable injection devices a medicament container, e.g., a cartridge has to be replaced when the medicament has been depleted. Here, the medicament container, e.g. a vitreous cartridge filled with the medicament has to be replaced. Some primary medicament containers, e.g. in form of a cartridge filled with the medicament are preassembled in a housing component of the injection device, such as a cartridge holder.

There further exist electronic units, e.g. either integrated into such delivery or injection devices or provided as a separate device, also denoted as a supplementary device or add-on device. Such supplementary devices provide additional functionality to an all-mechanically implemented drug delivery or injection device. By way of a supplementary device, repeated setting and dispensing or injecting of doses of the medicament can be precisely monitored and logged over time. Supplementary devices offer a large variety of supplemental functions for the regular use of drug delivery or injection devices. Supplementary devices may offer data analysis and further communication of such data to health care providers. Such data being indicative about the amount of a medicament administered at a particular point of time or at a particular date.

By way of electronically implemented supplementary devices, e.g. detachably mechanically connectable to a drug delivery or injection device handling and operation of such drug delivery devices can be facilitated.

In view of the above it is a further aim to enhance patient safety and to increase the functional scope of such supplementary devices, of drug delivery devices for use with such supplementary devices and to provide an improved injection system comprising a drug delivery device and a supplementary device.

### SUMMARY

In one aspect there is provided a supplementary device for attaching to a drug delivery device. The drug delivery device is operable to set and to dispense a dose of a medicament. Typically, the drug delivery device is implemented as an injection device, such as a handheld injection device. The drug delivery device may comprise a pen-type injector. The supplementary device is provided as a separate device detachably connectable to the drug delivery device. The supplementary device, also denoted as an add-on device provides additional function to the drug delivery device and offers to extend the functional scope of the drug delivery device.

The supplementary device comprises a fastener configured to fasten to a first component of the drug delivery device. The supplementary device further comprises a control element configured to engage with a second component of the drug delivery device. The control element typically engages or is engageable with the second component of the drug delivery device when the supplementary device is correctly attached, e.g. mechanically fastened to the first component of the drug delivery device.

The control element is movable relative to the fastener for inducing a movement of the second component of the drug delivery device relative to the first component of the drug delivery device when the fastener is attached to the first component. In effect, the first and the second components of the drug delivery device are movable relative to each other, e.g. for at least one of setting of the dose of the medicament and dispensing or injecting of the dose of the medicament.

Likewise, the fastener and the control element of the supplementary device are also movable relative to each other in the same way as the first and second components of the drug delivery device are movable relative to each other. When the first and the second components of the drug delivery device are movable e.g. along a longitudinal direction the control element is movable relative to the fastener also along the longitudinal direction. When the first and the second components of the drug delivery device are rotatable relative to each other the fastener may be also rotatable relative to the control element.

Typically, and when the supplementary device is correctly attached to the drug delivery device at least one of the fastener and the control element may cover and/or enclose at least one of the first component of the drug delivery device and the second component of the drug delivery device. Here, attachment of the supplementary device to the drug delivery device may impede or hinder direct access to at least one of the first and the second components of the drug delivery device. When for instance the fastener of the supplementary device is rigidly attached or fixed to the first component of the drug delivery device a manually operated or manually induced movement of the supplementary device relative to the drug delivery device, hence of the fastener of the supplementary device relative to the drug delivery device may invoke a movement of the first component of the drug delivery device, e.g. relative to a housing of the drug delivery device.

When correctly attached to the drug delivery device the control element of the supplementary device may cover and/or may be in mechanical engagement with the second component of the drug delivery device. Then and by manually inducing a motion or movement of the control element relative to the fastener there will be induced a respective motion of the second device component relative to the first device component. In this way and when the supplementary device covers or encloses at least one of the first and the second components of the drug delivery device it may still provide an indirect operation or manipulation of at least one of the first and the second components of the drug delivery device, via the respective engagement of the fastener with the first component, by the engagement of the control element with the second component and by the movability of the control element relative to the fastener.

The supplementary device further comprises an electronic unit. The electronic unit comprises at least a processor. The supplementary device further comprises a locking mechanism. The locking mechanism comprises an electromechanical actuator. The electromechanical actuator is controllable by the processor and is operable to block or to impede a movement of the control element relative to the fastener.

Typically, the locking mechanism is transferable between a release configuration and a locking configuration. In the release configuration the locking mechanism allows and supports a movement of the control element relative to the fastener, e.g. in order to induce a movement of the second component of the drug delivery device relative to the first component of the drug delivery device. When and as long as in the locking configuration the locking mechanism blocks or impedes at least a unidirectional movement of the control element relative to the fastener. In this way, and when a user should attempt to induce a movement of the second component of the drug delivery device relative to the first component of the drug delivery device, e.g. by moving, e.g. pushing, the control element relative to the fastener, the respective motion will be blocked and prevented as long as the locking mechanism is in the locking configuration. The user will then not be able to induce a respective movement of the second component of the drug delivery device.

Here, activation of the locking mechanism by the electromechanical actuator and/or by the processor actually controlling the electromechanical actuator blocks a respective movement of the control element relative to the fastener.

In a further aspect the supplementary device itself may be provided with an electrically or electronically implemented authentication function or feature by way of which authorized access to the supplementary device and hence authorized access to the drive unit can be electronically controlled. Hence, the supplementary device may be implemented to transfer the locking mechanism then into the release position or release configuration when a user intending to use the drive unit or drug delivery device has successfully passed an authentication procedure with the supplementary device.

Moreover, the supplementary device may be configured to lock automatically. It may be provided with a time lock. With some examples the supplementary device may be provided with patient specific data, e.g. being indicative of at least one of an amount of a medicament and a time or date when an injection is due. A user attempting to use the drive unit at any other time to administer a dose of inappropriate size may be effectively hindered to do so because the supplementary device may be configured to unlock the locking mechanism when a use of the drug delivery device user matches with a predefined prescription or medication schedule.

With further examples the authentication function conducted by the supplementary device may be operable to distinguish between different container units, e.g. equipped with different drug containers or medicament. Here, the drug delivery device, in particular a drive unit thereof may be configured to engage with a number of differently configured container units, e.g. that differ by the amount, the concentration and/or by the type of a drug or medicament. Each one of the container units being generally attachable to the drive unit may be provided with a unique identifier, e.g. by an electrically detectable or visual code or coding feature. Here, the supplementary device may be operable to read or to extract the relevant code information so as to obtain respective information about the drug or medicament provided in the container unit.

The container unit equipped with a medicament container may be visually or electronically encoded by a respective coding feature, e.g. implemented as a barcode, as a QR code or in form of an electronically readable code, e.g. provided by RFID chip. The supplementary device and/or an external electronic device operable to communicate with the supplementary device may be operable to capture and/or to read or to extract the coding feature of the container unit. Based on the captured coding feature at least one of the supplementary device and the external electronic device may evaluate the coding feature. The evaluation of the coding feature may include a comparison of the coding feature with user specific information, e.g. stored in one of the supplementary device and the external electronic device. If the coding feature matches with the user specific information, e.g. if it should be determined, that the type of medicament located inside the container unit matches a prescription schedule of a particular user, the locking mechanism should be transferred into the release configuration so as to allow and to support administering of a dose of the medicament.

In other cases and if it should be determined that the type of medicament does not match e.g. with a predefined prescription schedule of a particular user, the electronic unit, hence its processor is operable to keep the locking mechanism in a locked configuration so as to protect the patient against administering of a medicament that does not match with his prescription schedule. This way, misuse or unintended use of the drive unit can be then effectively prevented.

According to a further example the control element is movable relative to the fastener along a longitudinal direction between an idle position and an activation position. With some examples the drug delivery device is of elongated shape. It may extend along a longitudinal direction. Here, the supplementary device may be configured for attachment to a longitudinal end of the drug delivery device, typically at a proximal end of the drug delivery device, where the first component and the second component of the drug delivery device are located. Typically, a distal end of the drug delivery device is configured for expelling or injecting a dose of the medicament. When in the idle position the control element may be in mechanical engagement, e.g. in mechanical abutment with the second component of the drug delivery device.

Moving of the control element relative to the fastener, e.g. moving the control element in distal direction relative to the fastener may induce a distally directed movement of the second component of the drug delivery device relative to the first component of the drug delivery device. With some examples the control element can be pushed in distal direction relative to the fastener, thereby exerting a respective push action onto the second component. Pushing the second component in distal direction relative to the first component may induce or trigger a dispensing or injection procedure to be conducted by the drug delivery device.

According to a further example the control element comprises a first sidewall section. The fastener comprises a second side wall section. The first sidewall section is in a longitudinal sliding engagement with the second sidewall section. In this way the first and second side wall sections may provide a sliding guide for providing a controlled sliding displacement of the control element relative to the fastener.

With other examples the first and second side wall sections may enable or support a rotational motion of the control element relative to the fastener. Here, the first sidewall section may comprise a tubular structure and the second side wall section may comprise a complementary-shaped tubular structure.

In either way at least one of the first and the second side wall sections comprises an outside facing surface to slidably engage with an inside facing surface of the other one of the first and the second sidewall sections. In this way, the first and the second side wall sections can be arranged in a somewhat interleaved or nested manner. Hence, the control element can be telescopically moved relative to the fastener. The sliding engagement of first and second side wall sections provides a precise and guided movement of the control element relative to the fastener.

According to a further example the locking mechanism comprises a locking element mechanically coupled to the electromechanical actuator. The locking element is movable by the electromechanical actuator between a locking position and a release position. Movement of the locking element or a respective configuration of the locking element may define the operational status of the locking mechanism. Hence, when the locking element is in the locking position the locking mechanism is in a locking configuration. When the locking element is in the release position also the locking mechanism is in a release configuration.

The electromechanical actuator may comprise an electromechanical drive, an electromagnet or the like component in order to induce at least one of a bending, a movement or pivoting of the locking element induced by an electric current controlled by the electronic unit and its processor.

With a further example the locking element is movably arranged on one of the fastener and the control element. It is mechanically engageable with the other one of the fastener and the control element when in the locking position. Typically, the locking element may be provided on one of the first sidewall section and the second side wall section and may be operable to engage, e.g. to selectively engage with the other one of the first sidewall section and the second sidewall section. The engagement of the locking element with at least one of the fastener and the control element may comprise one of a positive fit and a friction fit. Moving of the locking element from the locking position towards and into the release position may abrogate the respective positive fit or friction fit.

With other examples the locking element may be provided on another component of the supplementary device, e.g. wherein the another component is fixed to one of the fastener and the control element. Then and by transferring the locking element into the release position it may mechanically interact or mechanically lock to one of the fastener and the control element.

With a further example of the supplementary device the locking element is movable along a radial direction or transverse direction between the locking position and the release position.

With further examples the locking element may be deformable along the radial direction to adapt to at least one of a locking shape and a release shape. When in the locking shape the locking element is in a locking configuration or locking position. When in the release shape, the locking element is in a release configuration or release position.

With some examples, the locking element may be elastically deformable. In an unbiased or idle position or configuration it may be in one of the locking position and the release position. By inducing a mechanical deformation of the locking element it may transfer or deform towards the other one of the locking position and the release position. In either way and since the locking element is movable and/or deformable in a radial or transverse direction it may selectively engage with at least one of a radial recess or radial protrusion in order to block or to impede movement of the control element relative to the fastener.

According to another example the locking element is configured to radially protrude from one of the first side wall section and the second side wall section. It may be further operable or configured to engage with a radial recess or with a longitudinal end face of the other one of the first side wall section and the second sidewall section. Typically, the locking element may be arranged in the interface between the first and the second sidewall sections. By moving the locking element in the radial or transverse direction it may protrude from one of an inside facing surface and an outside facing surface of one of the first and the second sidewall sections. It may then engage with the other one of an inside facing surface and an outside facing surface of the other one of first and the second sidewall sections.

In this way and when the locking element, e.g. shaped as a radial protrusion, engages with a radial recess there can be provided a positive or form fitting engagement between the first and the second sidewall portions and hence between the fastener and the control element. A further movement of the control element relative to the fastener may be effectively blocked as long as the protruding locking element is engaged with the radial recess.

With other examples the movable locking element may be provided with a radial recess and may be configured to engage with a complementary shaped protrusion. Here, the locking element may be movable in radial direction on one of the first and second sidewall sections and may thus engage with a protrusion provided on the other one of the first and the second sidewall sections.

According to a further example the locking element is mechanically coupled to the electromechanical actuator by a movable piston. The movable piston may comprise an elongated shaft. The elongated shaft may substantially extend along the radial or transverse direction. Actuation of the electromechanical actuator may therefore induce a movement of the movable piston in radial direction and may thus induces a respective radially or transversely directed movement of the locking element. The locking element may be provided or fixed at a longitudinal end of the movable piston.

According to a further example the locking element is rotatable or pivotable with regards to an axis extending along the longitudinal direction. A pivoting or rotating of the locking element may be also induced by the electromechanical actuator. Here, the electromechanical actuator may be operable to rotate the axis or a shaft overlapping with the axis. The locking element is attached to or mechanically coupled with the electromechanical actuator by way of the axis or shaft. Here, the electromechanical actuator may comprise a stepped motor to provide a precise angular rotation of the locking element.

According to a further example the locking element comprises a locking disc with a keyed structure extending along a circumference of the locking disc. The locking disc may be of circular or annular shape. With other examples the locking disc may comprise a radially outwardly extending flange connected to the axis or shaft.

The keyed structure may be configured and shaped to engage with a complementary shaped counter keyed structure. Typically, the locking element, e.g. the locking disc is longitudinally fixed to one of the fastener and the control element. The counter keyed structure is provided on the other one of the fastener and the control element. The plane of the locking disc may extend in a radial or transverse direction relative to the longitudinal direction and/or relative to the rotation axis of the rotatable locking element.

Then, a longitudinal sliding motion of the keyed or structure of the locking disc relative to the counter keyed structure is supported and enabled when the keyed structure correctly aligns with the counter keyed structure as seen in longitudinal direction. With the other orientations or rotational states of the locking disc, a respective longitudinal displacement of the locking disc and hence of the locking element can be effectively blocked.

With a further example, the locking element is arranged on one of the control element and the fastener. The other one of the control element and the fastener comprises a receptacle with a counter keyed structure complementary shaped to the structure of the locking element. Moving of the locking element into the receptacle is possible and allowed when the keyed structure is correctly aligned or is flush with the counter keyed structure. With the other orientations, the keyed structure will not match with the counter keyed structure and may thus impede or block insertion of the locking element into the receptacle. This way, a longitudinal sliding movement of the control element relative to the fastener can be effectively blocked and impeded.

According to a further example of the supplementary device the locking disc is arranged at a longitudinal offset from the counter keyed structure when the control element is in the idle position. The locking disc is then operable to block a longitudinal movement of the control element relative to the fastener because the keying structure is misaligned to the counter keyed structure.

Accordingly, and with a further example the control element is movable relative to the fastener towards the activation position when the keying structure of the locking disc is aligned with the counter keyed structure of the receptacle.

With some examples the keyed structure is provided on an outside facing surface of the locking disc. It may be located at an outer rim or outside circumference of the locking disc. The diameter or inside cross-section of the receptacle may be complementary shaped to the geometric structure of the keyed structure of the locking disc. With some examples the keyed structure comprises at least one radial protrusion and/or at least one radial recess. The counter keyed structure comprises a complementary shaped structure formed by at least one of a radial protrusion and a radial recess. Typically, the keyed structure comprises a protrusion configured to engage with a respective recess provided by the counter keyed structure. The counter keyed structure may comprise a number of elongated slots comprising a continuous profile as seen along the longitudinal direction. This allows for a longitudinal sliding movement of the keyed structure relative to the counter keyed structure.

With some examples the keyed structure comprises a series of teeth and recesses as seen along the outer circumference of the locking disc. Accordingly, the counter keyed structure of the receptacle comprises a respective series of counter teeth and counter recesses.

The electronic unit, in particular its processor and the electromechanical actuator are configured and operable to either align the keyed structure to the counter keyed structure or to misalign the keyed structure relative to the counter keyed structure. In a misaligned configuration the keyed structure is impeded to enter the recess in longitudinal direction. With a correctly aligned configuration, wherein the keyed structure is longitudinally flush with the counter keyed structure it will be possible to longitudinally displaceable the keyed structure into the recess, which motion is accompanied by a respective sliding movement of the control element relative to the fastener.

In another aspect the present disclosure also relates to a drug delivery system. The drug delivery system comprises a supplementary device as described above. The drug delivery system further comprises a drug delivery device. The drug delivery device may be implemented as an injection device, e.g. as a handheld pen-type injector. The drug delivery system may then comprise or constitute a drug injection system. The drug delivery device may be configured or operable to set and/or to dispense a dose of a medicament.

The drug delivery device comprises a container unit configured to accommodate a medicament container. The drug delivery device further comprises a drive unit operably engageable with the medicament container for setting and dispensing of a dose of the medicament. The drug delivery device further comprises at least one of a dose dial for setting of the dose and a trigger for dispensing of the dose. With some examples, wherein the drug delivery device comprises a dose dial and a trigger provided as separate components movable relative to each other, the fastener of the supplementary device may be configured to fix or to fasten to the dose dial and the control element may be operable to mechanically engage with the trigger.

With this or with other examples for inducing a drug delivery operation the control element may be displaceable relative to the fastener in longitudinal direction when the supplementary device is attached to the dose dial of the drug delivery device. A distally directed and user-induced movement of the control element relative to the fastener may then induce a respective displacement of the trigger relative to the dose dial.

With other examples and when the drug delivery device only comprises one user operable component, e.g. integrating the dose dial and the trigger in one mechanical component, e.g. denoted as a dosing element, the fastener of the supplementary device may be configured to fasten to a housing or housing component of the drug delivery device whereas the control element may be configured to engage or to fasten with the dosing element. The control element may be in a torque proof engagement with the dosing element and may be rotatable relative to the fastener thereby inducing a rotation of the dosing element relative to the housing or housing component when the supplementary device is appropriately fixed to the drug delivery device.

The control element may be also longitudinal displaceable relative to the fastener, e.g. in order to induce a respective distally directed displacement or motion of the dosing element relative to the housing of the drug delivery device, thereby triggering a dispensing or injecting action.

The locking mechanism may be operable to lock at least one of the rotational movement of the control element relative to the fastener and a longitudinal movement of the control element relative to the fastener.

With a further example the drug delivery device also comprises the medicament container, e.g. implemented as a cartridge, which is arranged inside the container unit. Typically, the drive unit of the drug delivery device comprises a piston rod to urge on a bung or stopper movably disposed in or on the medicament container for expelling the medicament, e.g. via a distal outlet of the medicament container.

The drive unit of the drug delivery device may be implemented mechanically. Here, a force for exerting or withdrawing a dose of the medicament from the medicament container may have to be entirely provided by a user of the device.

Generally, the scope of the present disclosure is defined by the contents. The injection device is not limited to specific embodiments or examples but comprises any combination of elements of different embodiments or examples. Insofar, the present disclosure covers any combination of embodiments and any technically feasible combination of the features disclosed in connection with different examples or embodiments.

It will be further apparent to those skilled in the art that various modifications and variations can be made to the present disclosure without departing from the scope of the disclosure.

In the present context the term ‘distal’ or ‘distal end’ relates to an end of the drug delivery device or injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-134-PC, PB-1023, TTP-054, Langlenatide/HM-1260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

### BRIEF DESCRIPTION OF THE DRAWINGS

In the following, numerous examples of a drive unit of a drug delivery device configured to interact with a supplementary device, of a drug delivery device, of a supplementary device and of a drug delivery system are illustrated and described in greater detail by making reference to the drawings, in which:

FIG. 1 illustrates a block diagram of the drug delivery system.

FIG. 2 schematically illustrates one example of a drug delivery device implemented as a handheld pen-type injector.

FIG. 3 shows a cross-section through an example of a supplementary device configured for attachment to the injection device.

FIG. 4 shows the supplementary device when attached to the injection device.

FIG. 5 shows an interlock mechanism of the supplementary device when in the locking configuration.

FIG. 6 shows the interlock mechanism of the supplementary device when in the release configuration.

FIG. 7 shows a further example of an interlock mechanism in the locking configuration.

FIG. 8 shows the example of FIG. 7 with the interlock mechanism in the release configuration.

FIG. 9 shows a cross section through one example of the interlock mechanism in a locking configuration.

FIG. 10 shows a cross section through the interlock mechanism of FIG. 9 when in the release configuration.

FIG. 11 shows a cross section through another example of the interlock mechanism in a locking configuration.

FIG. 12 shows a cross section through the interlock mechanism of FIG. 11 when in the release configuration.

### DETAILED DESCRIPTION

FIG. 2 shows an exemplary embodiment of a drug delivery device 1. The drug delivery device 1 is a variable dose device, in which different doses of a drug to be dispensed can be set or dialed, respectively, by a user. The drug delivery device is a dial extension pen.

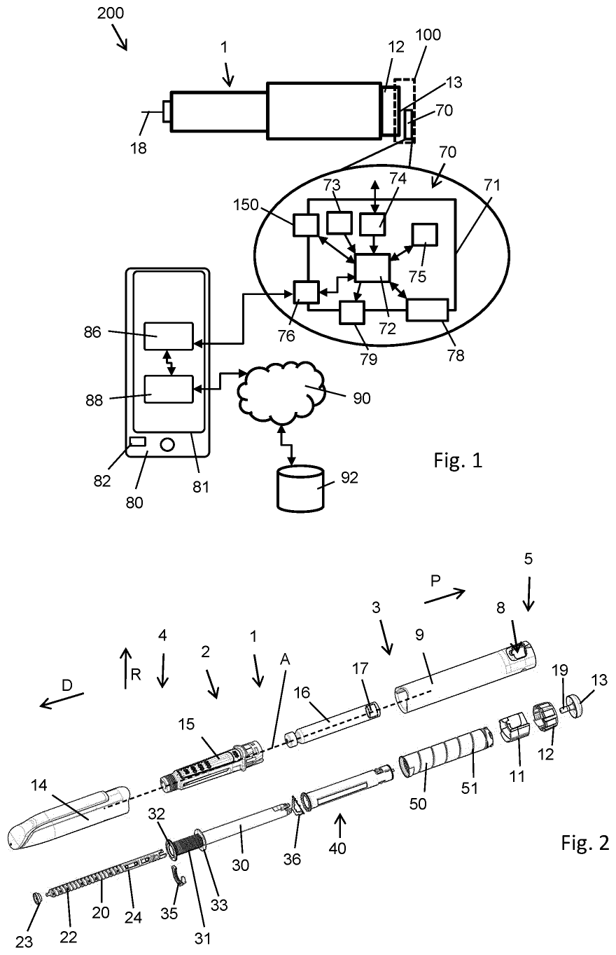

FIG. 1 also indicates the coordinate system used herein for specifying positions of members or elements or features. The distal direction D and proximal direction P run parallel to the longitudinal axis A. The longitudinal axis A is a main extension axis of the device 1. The radial direction R is a direction perpendicular to the longitudinal axis A and intersecting with the longitudinal axis A. A tangential direction, also referred to as circumferential direction is a direction perpendicular to the radial direction R and to the longitudinal axis A.

The drug delivery device 1 comprises a drive unit 3 with a setting mechanism and a dispense mechanism. The setting mechanism is configured for setting a drug dose and the dispense mechanism is configured for dispensing a drug dose. The functional principles of the mechanisms are explained further below.

The drug delivery device 1 further comprises a container unit 2 comprising a cartridge holder 15 configured and sized to accommodate a medicament container 16, e.g. in form of a cartridge. The medicament container 16 is sealed in proximal direction by a stopper 17. This particular drive unit 3 is exemplary for a large variety of drive units and injection devices for which the presently described supplementary device 100 can be used with. Further details about the specific implementation of the drive unit 3 can be found e.g. in WO2004/078239A1, WO 2004/078240A1 or WO 2004/078241A1 the entirety of which being incorporated herein by reference.

The drive unit 3 comprises a number sleeve 50 that is configured to move when a dose dial 12 is turned, in order to provide a visual indication of a currently set dose. The dose dial 12 is rotated on a helical path with respect to a housing 9 when turned during setting and/or dispensing or expelling of a dose. The housing 9 may comprise a window 8, e.g. in form of an aperture through which a portion of an outside surface of the number sleeve 50 is visible.

The injection device 1 may be configured so that turning the dose dial 12 causes a mechanical click sound to provide acoustical feedback to a user. The number sleeve 50 mechanically interacts with the stopper 17 in the cartridge or drug container 16. During delivery of dose, the dose dial 12 is turned to its initial position in an axial movement, that is to say without rotation, while the number sleeve 50 is rotated to return to its initial position, e.g. to display a dose of zero units. The injection device 1 may be implemented as a disposable injection device. The cartridge holder 15 is sized and configured to accommodate the drug container 16. With its proximal end it is non-detachably fastened to the proximal housing component 9, e.g. implemented as a body of the drive unit 3. The cartridge holder 15 is to be covered by protective cap 14 detachably fastened to the cartridge holder 15 and/or to the housing 9.

A flange like support of and/or inside the housing 9 comprises a threaded axial through opening threadedly engaged with a first thread or distal thread of a piston rod 20. The distal end of the piston rod 20 comprises a bearing on which a pressure foot 23 is free to rotate with the longitudinal axis of the piston rod 20 as an axis of rotation. The pressure foot 23 is configured to axially abut against a proximally facing thrust receiving face of the stopper 17 of the container 16. During a dispensing action the piston rod 20 rotates relative to the housing 9 thereby experiencing a distally directed advancing motion relative to the housing.

The piston rod 20 is provided with a first thread 22 at or near a distal end and is further provided with a second thread 24 at its proximal end. The first thread 22 and the second thread 24 are oppositely handed. There is further provided a drive sleeve 30 having a hollow interior to receive the piston rod 20. The drive sleeve 30 comprises an inner thread threadedly engaged with the proximal thread of the piston rod 20. Moreover, the drive sleeve 30 comprises an outer threaded section 31 at its distal end. The threaded section 31 is axially confined between a distal flange portion 32 and another flange portion 33, e.g., a proximal flange portion located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 9. In this way the last dose limiter 35 is splined to the housing 9. A rotation of the drive sleeve 30 in a dose incrementing direction or clockwise direction during consecutive dose setting procedures leads to an accumulative axial displacement of the last dose limiter 35 relative to the drive sleeve 30.

There is further provided an annular spring 36 that is in axial abutment with a proximally facing surface of the flange portion 33. Moreover, there is provided a tubular-shaped clutch 40. At a first end the clutch 40 is provided with a series of circumferentially directed saw teeth. Towards a second opposite end of the clutch 40 there is located a radially inwardly directed flange.

Furthermore, there is provided a dose dial sleeve also denoted as number sleeve 50. The number sleeve 50 is provided outside of the spring 36 and the clutch 40 and is located radially inward of the housing 9. A helical groove 51 is provided about an outer surface of the number sleeve 50. The housing 9 is provided with a dosage window through which a part of the outer surface of the number sleeve 50 can be visually inspected. The housing 9 is further provided with a helical rib 11 at an inside sidewall portion of an insert piece 10, which helical rib 11 is to be seated in the helical groove 51 of the number sleeve 50. The tubular shaped insert piece 10 is inserted into the proximal end of the housing 9. It is rotationally and axially fixed to the housing 9. There are provided first and second stops on the housing 9 to limit a dose setting procedure during which the number sleeve 50 is rotated in a helical motion relative to the housing 9.

The dose dial 12 in form of a dose dial grip is disposed about an outer surface of the proximal end of the number sleeve 50. An outer diameter of the dose dial 12 typically corresponds to and matches with the outer diameter of the housing 9 and may form or belong to the housing 9. The dose dial 12 may be rotationally secured to the number sleeve 50 to prevent relative movement therebetween. The dose dial 12 is provided with a central opening.

The trigger 13, also denoted as dose button is substantially T-shaped. It is provided at a proximal end of the injection device 1. It may form or constitute a proximal end of the housing 9. A shaft 19 of the trigger 13 extends through the opening in the dose dial 12, through an inner diameter of extensions of the drive sleeve 30 and into a receiving recess at the proximal end of the piston rod 20. The shaft 19 is retained for limited axial movement in the drive sleeve 30 and against rotation with respect thereto. A head of the trigger 13 is generally circular. A trigger side wall or skirt extends from a periphery of the head and is further adapted to be seated in a proximally accessible annular recess of the dose dial 12.

To dial a dose a user rotates the dose dial 12. With the spring 36 and the clutch 40 engaged, the drive sleeve 30, the spring 36, the clutch 40 and the number sleeve 50 rotate with the dose dial 12. Torque is transmitted through saw teeth between the spring 36 and the clutch 40. The helical groove 51 on the number sleeve 36 and a helical groove in the drive sleeve 30 have the same lead. This allows the number sleeve 50 to extend from the housing 9 and the drive sleeve 30 to climb the piston rod 20 at the same rate. At a limit of travel a radial stop on the number sleeve 50 engages either with a first stop or a second stop provided on the housing 9 to prevent further movement in a first sense of rotation, e.g. in a dose incrementing direction. Rotation of the piston rod 20 is prevented due to the opposing directions of the overall and driven threads on the piston rod 20.

The last dose limiter 35 keyed to the housing 9 is advanced along the threaded section 31 by the rotation of the drive sleeve 30.

During dialing or setting of a dose a ratchet mechanism allows and supports a rotation of the number sleeve 50 relative to the clutch 40 along a second sense of rotation, which rotation is accompanied by a regular clicking of a flexible arm of the clutch 40. An angular momentum applied to the number sleeve 50 along the first sense of rotation is unalterably transferred to the clutch 40. Here, the mutually corresponding ratchet features of the ratchet mechanism provide a torque transmission from the number sleeve 50 to the clutch 40.

When the desired dose has been dialed the user may simply dispense the set dose by depressing the trigger 13. This displaces the clutch 40 axially with respect to the number sleeve 50 causing dog teeth thereof to disengage. However, the clutch 40 remains keyed in rotation to the drive sleeve 30. The number sleeve 50 and the dose dial 12 are now free to rotate in accordance with the helical groove 51.

The axial movement deforms the flexible arm of the spring 36 to ensure the saw teeth cannot be overhauled during dispense. This prevents the drive sleeve 30 from rotating with respect to the housing 9 though it is still free to move axially with respect thereto. The deformation is subsequently used to urge the spring 36 and the clutch 40 back along the drive sleeve 30 to restore the connection between the clutch 40 and the number sleeve 50 when the distally directed dispensing pressure is removed from the trigger 13.

The longitudinal axial movement of the drive sleeve 30 causes the piston rod 20 to rotate through the through opening of the support of the housing 9, thereby to advance the stopper 17 in the container 16 and to expel a dose of the medicament via an injection needle 18 attached to the distal end of the injection device. Once the dialed dose has been dispensed, the number sleeve 50 is prevented from further rotation by contact of at least one stop extending from the dose dial 12 with at least one corresponding stop of the housing 9. A zero dose position may be determined by the abutment of one of axially extending edges or stops of the number sleeve 50 with at least one or several corresponding stops of the housing 9.

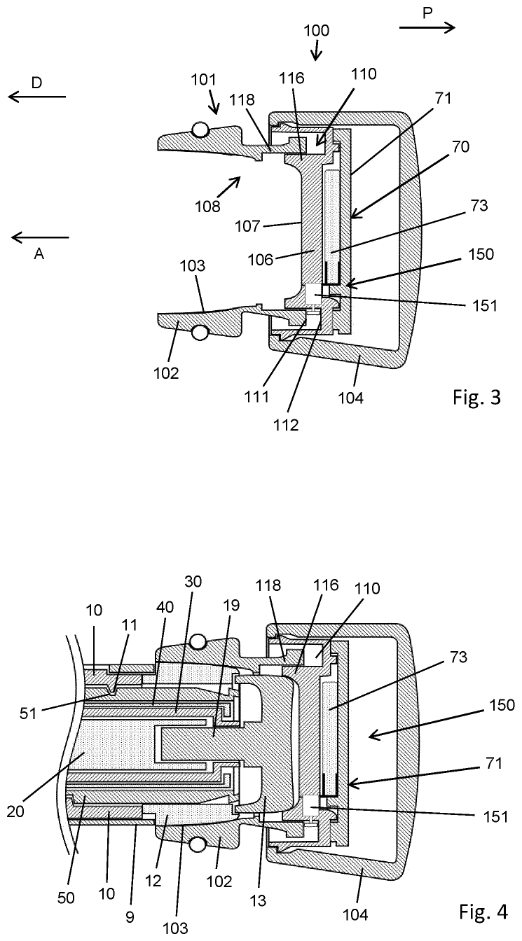

In FIGS. 3-6 one example of a supplementary device 100 configured for attachment to the proximal end 5 of the drive unit 3 is schematically illustrated. The supplementary device 100 comprises a housing 101. The housing 101 comprises a first housing portion 102 and a second housing portion, e.g. in form of a control element 104 movably disposed on the housing portion 102.

The housing portion 102 comprises or forms a fastener 103 configured and shaped to detachably fasten to the proximal end of the drive unit 3. As illustrated in FIGS. 3 and 4, the fastener 103 is provided on an inside surface of the somewhat tubular shaped housing component 102. The housing component 102 forms or constitutes a receptacle 108 sized and shaped to engage with an outside surface of the dose dial 12.

Accordingly, the fastener 103 may be configured and operable to form at least one of a form fit and a friction fit with the outside surface of the sidewall of the dose dial 12. The dose dial 12 may be provided with longitudinal ribs, which may support a torque proof engagement of the fastener 103 to the dose dial 12. Here, the dose dial 12 represents a first component of the drug delivery device 1.

The control element 104, hence a second housing component movably arranged on the housing component 102 comprises a knob-like shape. It comprises an interior hollow space, in which there is arranged an electronic unit 70. The control element 104 comprises a receptacle sized and shaped to receive at least a part of the housing portion 102. As illustrated in greater detail in FIG. 3, the housing portion 102 comprises a second tubular sidewall section 118 engaged with a respective first sidewall section 116 of the control element 104. Here, the sidewall section 116 is permanently fixed to the control element 104 or may be integrated into the control element 104.

The second sidewall section 118 is in longitudinal sliding engagement with the first sidewall section 116. The first side wall section 116 may be provided on a carrier 106 rigidly fastened to the control element 104. The carrier 106 may enclose the cup-shaped receptacle of the control element 104 towards the distal direction D. The carrier 106 comprises a distally facing surface 108 effectively closing the receptacle 108 of the housing portion 102 towards the proximal direction P.

The carrier 106 is configured to mechanically support the electronic unit 70. The electronic unit 70 comprises a processor 72, a printed circuit board (PCB) 71, a battery 73. The electronic unit 70 may optionally comprise a sensor 74, a storage 75, a communication interface 76, a display 78 and a signal generator 79. The electronic unit 70 is operably coupled to a locking mechanism 150 comprising an electromechanical actuator 151. Details of the electronic unit will be explained below in connection with FIG. 1.

In FIG. 4, there is illustrated a drug delivery system 200, wherein the supplementary device 100 is detachably fastened to the proximal end 5 of the drug delivery device 1. The drug delivery system 200 comprises the supplementary device 100 and the drug delivery device 1.

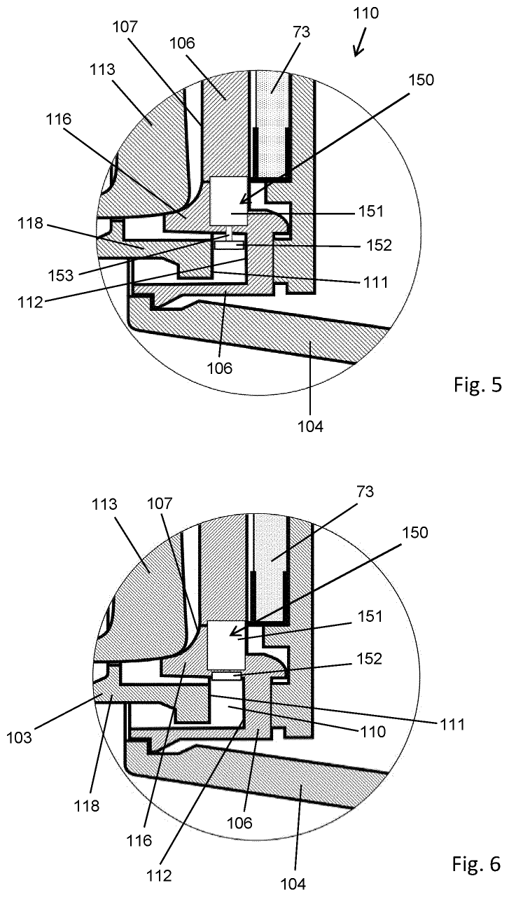

The supplementary device 100 comprises the locking mechanism 150 featuring the electromechanical actuator 151. The electromechanical actuator 151 is operable or controllable by the electronic unit 70, hence by the processor 72 the electronic unit 70. The locking mechanism 150 is transferable between a locking configuration as illustrated in FIG. 5 and a release configuration as shown in FIG. 6. In the locking configuration the locking mechanism 150 is operable to block or to impede a longitudinal sliding movement of the control element 104 relative to the fastener 103.

Typically, and in order to dispense a dose, a distally directed push force can be applied onto the control element 104 so as to move the control element 104 in distal direction relative to the fastener 103. Since the fastener 103 is rigidly attached to the dose dial 12 a respective distally directed movement of the control element 104 serves to induce a distally directed force effect onto the trigger 13 or trigger button, thereby triggering or controlling a dispensing action of the drive unit 3.

As shown in greater detail in FIGS. 5 and 6 the locking mechanism 150 is provided with a locking element 152, which is mechanically connected to the electromechanical actuator 151 by a piston 153. The piston 153 and hence the locking element 152 is movable by the electromechanical actuator 151 between a radially recessed or retracted position as shown in FIG. 6 and a radially protruding position as shown in FIG. 5. When in the protruding position as shown in FIG. 5 the locking element 152 is located inside a free space 110 confined in distal direction by an end face 111 of the second sidewall portion 118 and being further confined in proximal direction by a stop face 112 of the carrier 106 and hence by the control element 104.

The longitudinal size of the blocking element 152 substantially matches the longitudinal extent of the free space 110. In this way and when the locking element 152 is in the radially extended position the electromechanical actuator is in a locking position or blocking configuration. The presence of the locking element 152 in the free space 110 or in the recess formed between the faces 111, 112 impedes and blocks a distally directed movement of the carrier 106 and hence of the control element 104 in distal direction relative to the fastener 103.

Accordingly, and when a user should attempt to apply a dispensing force onto the control element 104 in distal direction such dispensing force will be inoperable to induce a movement of the control element 104 relative to the fastener 103. Accordingly, the user will be hindered to depress the trigger 13 in distal direction D. It is upon transferring of the electromechanical actuator 151 into the release position as shown in FIG. 6 to remove the locking element 152 from the free space 110 between the end face 111 and the stop face 112.

Then the control element 104 and its carrier 106 is no longer hindered to move in distal direction as a user applies a respective distally directed pressure onto the control element 104. Accordingly, the first sidewall section 116 and hence the control element 104 is allowed to slide along the second sidewall section 118 of the fastener 103 in longitudinal direction.

The distally facing surface 107 of the carrier 106 is then operable to apply a respective distally directed pressure onto the trigger 13 being effective to depress the trigger for dispensing of a dose.

With other examples it is also conceivable, that an inside facing surface of the second sidewall section 118 comprises a recess, e.g. a radial recess shaped and configured to engage with the locking element 152. As the locking element 152 is in the locking position the control element 104 is hindered to leave its idle position as illustrated in FIG. 5. Retraction of the locking element 152 out of a respective radial recess of the second sidewall section 118 will then lead to a respective disengagement of first and second sidewall sections 116, 118 and thus allows to move the control element 104 from the idle position into a distally displaced activation position.

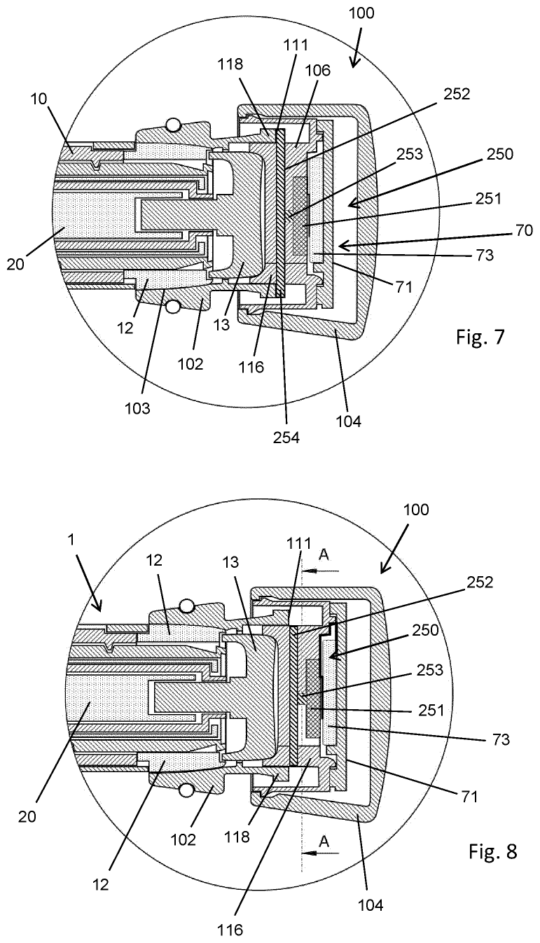

In FIGS. 7-12 there are illustrated two further examples of a supplementary device 100 equipped with a different kind of a locking mechanism 250. The other parts of the supplementary device 100 according to FIG. 7-12 are implemented in the same way as described before in connection with FIGS. 3-6.

The locking mechanism 250 also comprises an electromechanical actuator 251. But here, the actuator 251 one is mechanically coupled to an axis 253 extending along the longitudinal direction A. The axis 253 serves as a longitudinally extending shaft and is connected with a locking disc 255. The locking disc 255 is rotatable relative to the axis 253 through actuation of the electromechanical actuator 251. Here, the electromechanical actuator 251 may be implemented as a stepper motor and is thus operable to rotate the locking disc 255 with regard to the axis 253.

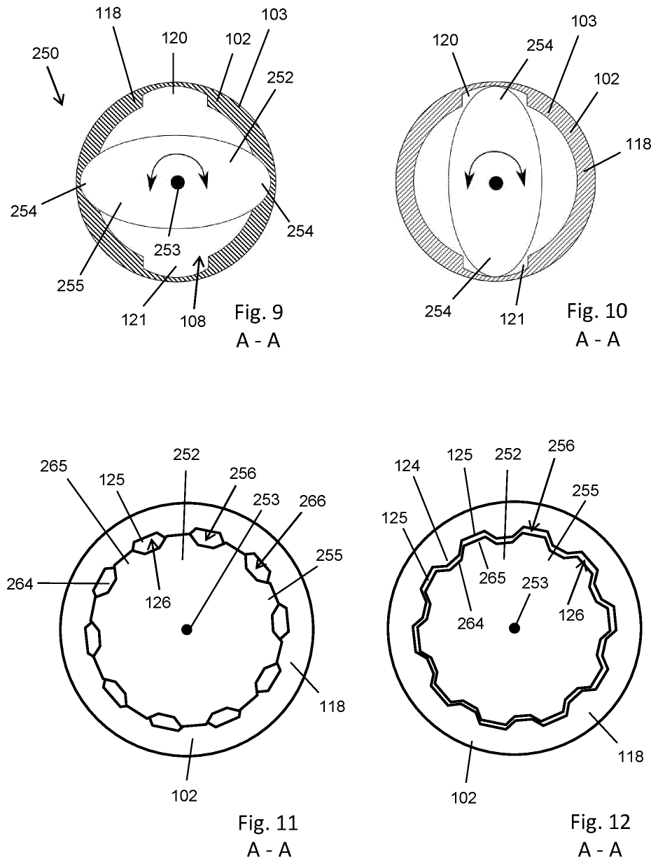

As illustrated in FIGS. 7 and 8, both representing the idle position of the fastener 104, the locking element 252, in particular the locking disc 254 is located at a longitudinal offset from the second sidewall section 118 of the fastener 103. A distally facing surface of the locking element 252 and hence of the locking disc 255 may be in axial abutment with a proximal end face 111 of the second sidewall section 118. As illustrated for instance in FIGS. 9 and 10 the locking disc 255 is of elliptical shape. The locking disc 255 may thus comprise two oppositely located longitudinal end sections 254 that are located at a radial distance that is larger than an inner diameter of the tubular shaped second sidewall section 118.

In the configuration as illustrated in FIGS. 7 and 9 the oppositely located longitudinal end sections 254 are out of alignment with complementary shaped longitudinal slots 120, 121 located diametrically opposite on the inside surface of the second sidewall section 118. In this configuration the geometric shape and orientation of the locking disc 255 non-matching with the inside or geometry of the second sidewall portion 118 prevents a longitudinal insertion of the locking disc 255 and hence of the locking element 252 into a free space circumferentially confined by the second sidewall section 118.

Activating the electromechanical actuator 251 may serve to rotate the locking element 252 with the axis 253 as an axis of rotation. When reaching a release position as illustrated in FIGS. 8 and 10 the oppositely located longitudinal end sections 254 of the elliptically shaped locking disc 255 may align with the slots 120, 121. The inside distance of the sidewall section 118 in the region between the two slots 120, 121 is equal to or slightly larger than an outside distance between the oppositely located longitudinal end sections 254 of the locking disc 255.

In this configuration it is now possible to move the locking disc 255 in longitudinal direction into the interior space of the second sidewall section 118. Hence, the keyed structure of the locking element 252 is correctly aligned with the counter keyed structure of the second sidewall section 118 and a longitudinal and distally directed sliding motion of the locking element 252 and hence a respective longitudinal displacement of the first sidewall section 116 of the control element 104 relative to the second sidewall section 118 of the fastener 103 is allowed and supported.

With the further example of FIGS. 11 and 12 the locking disc 255 is provided with a keyed structure 256 at its outside circumference. The keyed structure 256 comprises a number of recesses 264 and protrusions 265. There is provided an alternating arrangement of recesses 264 and respective protrusions 265 as seen along the circumference of the locking disc 255. An inside surface of the second sidewall section 118 is provided with a complementary shaped counter keyed structure 126. The counter keyed structure 126 also comprises a number of radially inwardly protruding protrusions or teeth 124 and recesses 125 also arranged in an alternating arrangement on or along the inside circumference of the second sidewall section 118.

In the locking position or blocking configuration as shown in FIG. 11 the protrusions 265 of the keying structure 256 are aligned with the protrusions or teeth 124 of the counter keyed structure 126. In this non-matching alignment or configuration the keyed structure 256 is in a blocking engagement with the counter keyed structure 126. By rotating the locking disc 255 through actuation of the electromechanical actuator 251 with the axis 253 as an axis of rotation the protrusions 265 of the keyed structure 256 may align with the recesses 125 of the counter keyed structure 126.

Accordingly, the teeth 124 of the counter keyed structure 126 may align with the recesses 264 of the keyed structure 256. Likewise and with this type of a matching keyed alignment of the keyed structure 256 with the counter keyed structure 126 the locking mechanism 250 is in the release configuration and the control element 104 is longitudinally, e.g. distally displaceable relative to the fastener 103 in order to trigger a dose dispensing or dose injection action.

FIG. 1 is a schematic block diagram of the drug delivery system 200 comprising the drug delivery device 1 and is provided with a supplementary device 100.

The supplementary device 100 is inter alia equipped with a printed circuit board (PCB) 71. There is provided a processor 72 on the PCB. The processor 72 may for instance be a microprocessor, a Digital Signal Processor (DSP), Application Specific Integrated Circuit (ASIC), Field Programmable Gate Array (FPGA) or the like. Processor 72 is configured and operable to execute program code (e.g. software or firmware) stored in a storage 75. The storage 75 may comprise a program memory and may use a main memory, for instance to store intermediate results. The storage 75 may also be used to store a logbook on performed ejections/injections. The storage 75, hence the program memory may for instance be a Read-Only Memory (ROM), and main memory may for instance be a Random Access Memory (RAM).

With some examples, processor 72 interacts with an input, e.g. in form of one or a plurality of buttons or actuators by way of which the supplementary device 100 may for instance be turned on and off. A further button may be implemented as a communications button. The further button may be used to trigger establishment of a communication link or data connection to an external electronic device 80, which is e.g. implemented as a smart phone. Still another button may be implemented as a confirmation button or OK button. This button may be used to acknowledge information presented to a user of the supplementary device 100. The buttons may be any suitable form of user input transducers, for instance mechanical switches, capacitive sensors or other touch sensors.

The processor 72 may control a display 78 or display unit, which is presently embodied as a Liquid Crystal Display (LCD). The optional display 78 is operable to display information to a user of the supplementary device 100, for instance on present settings of injection device 1 or on a next injection to be conducted. The display 78 or display unit may also be embodied as a touch-screen display, for instance to receive user input.

The processor 72 may be further operationally coupled to a sensor 74. The sensor 74 may be implemented as an optical sensor, embodied as an Optical Character Recognition (OCR) reader, that is capable of capturing images of the window 12 or dosage display, in which a currently selected dose is displayed (by way of numbers printed on the sleeve 26 contained in injection device 1, which numbers are visible through the window 12 or dosage display). The OCR reader may be further capable of recognizing characters (e.g. numbers) from the captured image and to provide this information to the processor 72.

Alternatively, the sensor 74 of the supplementary device 100 may be an optical sensor, e.g. a camera, for capturing images and providing information on the captured images to processor 72. Then, the processor 72 might by operable to perform OCR on the captured images.

The processor 72 may also control light-sources such as light emitting diodes to illuminate the window 12, in which a currently selected dose is displayed. A diffuser may be used in front of the light-sources, for instance a diffuser made from a piece of acrylic glass. Furthermore, the optical sensor 74 may comprise a lens (e.g. an aspheric lens) leading to a magnification (e.g. a magnification of more than 3:1).

The sensor 74 may be also implemented as an acoustic sensor, which is configured to sense sounds produced by the injection device 1. Such sounds may for instance occur when a dose is dialed, e.g. by turning the dose dial 12 or dose button 13, and/or when a dose is ejected/injected by the dose button 13 or trigger 13, and/or when a prime shot is performed. These actions are mechanically similar but nevertheless sound differently (this may also be the case for electronic sounds that indicate these actions). Here, either the sensor 74 and/or processor 72 may be configured to differentiate these different sounds, for instance to be able to safely recognize that an injection has taken place (rather than a prime shot).

The processor 72 may further control a signal generator 79, e.g. implemented as an acoustic signal generator which is configured to produce acoustical signals that may for instance be related to the operating status of injection device 1, for instance as a feedback to the user. For example, an acoustical signal may be launched by the signal generator 79 as a reminder for the next dose to be injected or as a warning signal, for instance in case of misuse. Acoustical signal generator may for instance be embodied as a buzzer or loudspeaker. In addition to or as an alternative to acoustical signal generator 79, also a haptic signal generator (not shown) may be used to provide haptic feedback, for instance by way of vibration.

The processor 72 further controls a communication interface 76, e.g. implemented as a wireless unit or as a wireless transceiver. The communication interface 76 is configured to transmit and/or to receive information to or from another device in a wireless fashion. Such transmission may for instance be based on radio transmission or optical transmission. Typically, the communication interface 76 is implemented pursuant to commercially available wireless communication standards, such as Bluetooth, Wi-Fi, UWB or NFC.

Alternatively, the communication interface 76 may comprise a wired unit configured to transmit and/or receive information to/from another device in a wire-bound fashion, for instance via a cable or fibre connection. When data is transmitted, the units of the data (values) transferred may be explicitly or implicitly defined. For instance, in case of an insulin dose, International Units (IU) may be used, or otherwise, the used unit may be transferred explicitly, for instance in coded form.

The processor 72 may be operable to receive an input from a pen detection switch (not illustrated), which is operable to detect whether the pen 1 or drug delivery is present, e.g., to detect whether the supplementary device 100 is correctly coupled to the injection device 1. A battery 73 powers the processor 72 and other components of the electronica unit 70.

The supplementary device 100 is thus capable of determining information related to a condition and/or use of injection device 1. This information may be displayed on the display 21 for use by the user of the device. The information may be either processed by supplementary device 100 itself, or may at least partially be provided to an external electronic device 80 for further data processing.

Generally, and by way of the sensor 74 information regarding the size of the dose currently set, dialed or dispensed can be obtained and can be stored in a local storage 75. Data repeatedly captured or obtained during repeated and subsequent use of the injection device 1 can be stored in the storage 75. Such injection-related data stored in the storage 75 can be synchronized, e.g. by the communication interface 76 communicating with the external electronic device 80 and/or with an external database 96, e.g. hosted or provided by a healthcare provider.

Typically, the external electronic device 80 is implemented as a smart phone, as a smart watch or as a tablet computer. It may comprise a wide range communication interface 88 to establish a communication link to the external database 92 via a communication network 90. Typically, the external electronic device 80 is a mobile device or wearable device. It comprises a display 81 and/or a speaker 82 to communicate with a user.

The external electronic device 80 may be wirelessly paired to the electronic unit 70. Upon successful pairing a respective confirmation may be provided to a user, e.g. via the display 81 and/or via the speaker 82 of the external electronic device 80. Upon transferring of data or information between the electronic unit 70 and the external electronic device 80 a visual and/or audible confirmation may be provided to the user, e.g. by the electronic unit 70 and/or by the external electronic device 80.

The processor 72 may be configured to implement an automatic re-locking, e.g. after termination of each dispensing or in injecting procedure. Moreover, and based on signals obtainable from the sensor 74 the processor 72 may be provided with information being indicative of the remaining filling level of the medicament container 16. In this way and when the sensor 74 provides respective sensor signals that the medicament container 16 is substantially depleted or empty the processor 72 may control and/or actuate the release mechanism 250, 350 and thus the actuator 251, 351 to transfer the mechanical locking element 51, 151 into the locking position.

With further examples the electrically controlled release mechanism 250, 350 of the supplementary device 100 may be controlled by the processor 72 on the basis of an authentication routine conducted by the supplementary device 100 and/or conducted by the external electronic device. This may particularly apply with reusable injection devices 1 having a common drive unit 3 but distinguish by their container unit 2 and/or by the medicament stored in the medicament container 16.

Here, a particular container unit 2 equipped with a medicament container 16 may be visually or electronically encoded by a respective coding feature, e.g. implemented as a barcode, as a QR code or in form of an electronically readable code, e.g. provided by RFID chip. At least one of the supplementary device 100 and the external electronic device 80 may be operable to capture and/or to read or to extract the coding feature of the container unit 2. Based on the captured coding feature the processor 72 may be operable to activate or to deactivate the locking mechanism 150, 250. If a wrong container unit 2 should be paired and/or connected to the drive unit 3, the coding feature captured and/or processed by the processor 72 will be inoperable to activate the interlock mechanism 150, 250 for switching the mechanical locking element 152, 252 into the release position. Then, the locking mechanism 50, 150 may remain in the locked position or locked configuration. Misuse or unintended use of the drive unit 3 is then effectively prevented.

The electronic unit 70, in particular its processor 72 may be operable to activate the interlock release 150, 250 when a correct coding feature is detected. The evaluation of a correct coding feature may be conducted by the processor 72 and/or by a processor of the external electronic device 80. Here, approval of an authentication to unlock the locking mechanism 150, 250 may be also conducted by the external electronic device 80.

### REFERENCE NUMBERS

1 drug delivery device
2 container unit
3 drive unit
4 distal end
5 proximal end
8 window
9 drive unit housing
10 insert piece
11 helical rib
12 dose dial
13 trigger
14 protective cap
15 cartridge holder
16 medicament container
17 stopper
18 injection needle
19 shaft
20 piston rod
21 bearing
22 first thread
23 pressure foot
24 second thread
30 drive sleeve
31 threaded section
32 flange
33 flange
35 last dose limiter
36 spring
40 clutch
50 number sleeve
51 groove
70 electronic unit
71 printed circuit board
72 processor
73 battery
74 sensor
75 storage
76 communication interface
78 display
79 signal generator
100 supplementary device
101 housing
102 housing portion
103 fastener
104 control element
106 carrier
107 surface
108 receptacle
110 free space
111 end face
112 stop face
116 sidewall section
118 sidewall section
120 slot
121 slot
124 tooth
125 recess
126 counter keyed structure
150 locking mechanism
151 actuator
152 locking element
153 piston
200 drug delivery system
250 locking mechanism
251 actuator
252 locking element
253 axis
254 end section
255 locking disc
256 keyed structure
264 recess
265 protrusion

1-18. (canceled)

19. A supplementary device for attaching to a drug delivery device, wherein the drug delivery device is operable to set and dispense a dose of a medicament, the supplementary device comprising:
a fastener configured to fasten to a first component of the drug delivery device;
a control element configured to engage with a second component of the drug delivery device, wherein the control element is configured to be movable relative to the fastener for inducing a movement of the second component relative to the first component when the fastener is attached to the first component;
an electronic unit comprising a processor; and
a locking mechanism comprising an electromechanical actuator controllable by the processor and operable to block or impede a movement of the control element relative to the fastener.

20. The supplementary device according to claim 19, wherein the control element is movable relative to the fastener along a longitudinal direction between an idle position and an activation position.

21. The supplementary device according to claim 19, wherein the locking mechanism comprises a locking element mechanically coupled to the electromechanical actuator and movable by the electromechanical actuator between a locking position and a release position.

22. The supplementary device according to claim 21, wherein:
the locking element is movably arranged on one of the fastener and the control element and is mechanically engageable with the fastener and the control element when in the locking position.

23. The supplementary device according to claim 21, wherein the locking element is movable along a radial direction or transverse direction between the locking position and the release position.

24. The supplementary device according to claim 21, wherein:
the control element comprises a first sidewall section,
the fastener comprises a second sidewall section, and
the first sidewall section is in longitudinal sliding engagement with the second sidewall section.

25. The supplementary device according to claim 24, wherein the locking element is configured to radially protrude from one of the first sidewall section or the second sidewall section to engage with a radial recess of the other one of the first sidewall section and the second sidewall section.

26. The supplementary device according to claim 24, wherein the locking element is configured to radially protrude from one of the first sidewall section or the second sidewall section to engage with a longitudinal end face of the other one of the first sidewall section and the second sidewall section.

27. The supplementary device according to claim 21, wherein the locking element is mechanically coupled to the electromechanical actuator by a movable piston.

28. The supplementary device according to claim 21, wherein the locking element is rotatable or pivotable relative to an axis extending along the longitudinal direction.

29. The supplementary device according to claim 21, wherein the locking element comprises a locking disc with a keyed structure extending along a circumference of the locking disc.

30. The supplementary device according to claim 29, wherein the locking element is arranged on one of the control element and the fastener and wherein the other one of the control element and the fastener comprises a receptacle with a counter keyed structure complementary shaped to the keyed structure.

31. The supplementary device according to claim 30, wherein:
the locking disc is arranged at a longitudinal offset from the counter keyed structure when the control element is in an idle position, and
the locking disc is operable to block a longitudinal movement of the control element relative to the fastener towards an activation position when the keyed structure is misaligned to the counter keyed structure.

32. The supplementary device according to claim 30, wherein the control element is movable relative to the fastener towards an activation position only when the keyed structure of the locking disc is aligned with the counter keyed structure of the receptacle.

33. The supplementary device according to claim 19, wherein the drug delivery device comprises a dose dial for setting of the dose and a trigger for dispensing of the dose, wherein the dose dial and the trigger are separate components being movable relative to each other, wherein the fastener is configured to fix or to fasten to the dose dial and the control element is operable to mechanically engage with the trigger.

34. The supplementary device according to claim 19, wherein the control element is displaceable relative to the fastener in a longitudinal direction for inducing a drug delivery operation when the supplementary device is attached to a dose dial of the drug delivery device.

35. A drug delivery system comprising:
a drug delivery device for setting and dispensing of a dose of a medicament, the drug delivery device comprising:
a container unit configured to accommodate a medicament container;
a drive unit operably engageable with the medicament container for setting and dispensing of a dose of the medicament; and
at least one of a dose dial for setting of the dose and a trigger for dispensing of the dose; and
a supplementary device comprising
a fastener configured to fasten to one of the dose dial and the trigger,
a control element configured to engage with the other of the dose dial and the trigger, wherein the control element is configured to be movable relative to the fastener for inducing a movement of the dose dial and the trigger relative to one another when the fastener is fastened to the one of the dose dial and the trigger;
an electronic unit comprising a processor; and
a locking mechanism comprising an electromechanical actuator controllable by the processor and operable to block or impede a movement of the control element relative to the fastener.

36. The drug delivery system according to claim 35, wherein the fastener is configured to fasten to the dose dial, and the control element of the supplementary device is operable to mechanically engage with the trigger.

37. The drug delivery system according to claim 35, wherein the drug delivery device further comprises the medicament container arranged inside the container unit.

38. The drug delivery system according to claim 35, wherein:
the drug delivery system comprises both the dose dial and the trigger,
the dose dial and the trigger are separate components being movable relative to each other, and
the fastener is configured to fasten to the dose dial and the control element of the supplementary device is operable to mechanically engage with the trigger.
